(19) United States
(12) Patent Application Publication
(10) Pub. No.: US 2006/0013869 A1
(43) Pub. Date: Jan. 19, 2006

(54) Electrospun amorphous pharmaceutical compositions

(76) Inventors: Francis Ignatious, King of Prussia, PA (US); Linghong Sun, Collegeville, PA (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 10/523,835

(22) PCT Filed: Aug. 7, 2003
(86) PCT No.: PCT/US03/24641

§ 371 Date: Feb. 7, 2005

Related U.S. Application Data
(60) Provisional application No. 60/401,726, filed on Aug. 7, 2002.

(51) Int. Cl. A61K 9/20

(52) U.S. Cl. 424/464

(57) ABSTRACT

The present invention is directed to use of electrospinning, i.e. the process of making polymer nanofibers from either a solution or melt under electrical forces, to prepare stable, solid dispersions of amorphous drugs in polymer nanofibers.

### FIELD OF THE INVENTION

This invention relates to stabilization of solid dispersions of amorphous drugs in polymeric nanofibers, method of preparation thereof and pharmaceutical compositions containing these nanofibers.

### BACKGROUND

With the advent of combinatorial chemistry and high throughput screening, a great majority of the drug candidates selected for development are highly hydrophobic, exhibiting poor or negligible water solubility. In order to enhance the oral absorption of such poorly water soluble drugs, several formulation strategies such as salt formation, complexation, particle size reduction, prodrug, micellization, and solid dispersions are being extensively studied in the pharmaceutical industry.

Although solid dispersions have been known for the past four decades, there seems to be renewed interest in this technology, as described by Serajudin et al., Journal of Pharmaceutical Sciences, 1999, 88 (10), 1058 and by Habib et al., Pharmaceutical Solid Dispersion Technology, (Technomic, Lancaster, Pa., 2001). Solid dispersions may be defined as the dispersion of one or more active ingredient in an inert carrier or matrix in the solid state prepared by the melting method, the solvent method or the melting-solvent method. Solid dispersions are classified into six major categories: (1) simple eutectic mixtures (2) solid solutions, (3) glass solutions of suspensions, (4) amorphous precipitation of a drug in a crystalline carrier, (5) amorphous precipitation of a drug in a amorphous carrier, and (6) any combination of these groups.

Two currently used methods of forming solid dispersions are fusion and solvent methods. In the fusion method, the drug and the carrier are melted, to above either the melting (softening) point of the higher melting (softening) component, or in some cases to above the melting point of the lower melting component provided the other non-melted component has good solubility in the former. The fused mixture is rapidly quenched and pulverized to produce free flowing powders for capsule filling or tableting. The fusion process requires both the drug and excipient to be thermally stable at the processing temperature.

In the solvent method, the drug and carrier are dissolved in one or more miscible organic solvents to form a solution. Removal of the organic solvent(s) is accomplished by any one or a combination of methods such as solvent evaporation, precipitation by a non-solvent, freeze drying, spray drying, and spray congealing. Among the several draw backs of the solvent method are: use of large volumes of organic solvents, presence of residual organic solvents in the resultant formulation, collection, recycling and/or disposal of organic solvents.

Solid dispersions of poorly soluble drugs prepared by both the fusion and solvent methods usually exhibit higher dissolution rates than the comparative crystalline drug. However, the dissolution rate of the drug may be hindered by dissolution of the carrier, usually a high molecular weight polymer. Therefore solid dispersions are usually prepared from low or moderate molecular weight polymers.

The need still remains to develop a process by which solid dispersions can be made of drugs having an amorphous morphology, that remain stable, and can use higher molecular polymers to aid in the dissolution rates of these drugs.

### BRIEF DESCRIPTION OF THE DRAWINGS

FIG. 1 demonstrates a schematic representation electrospinning of viscous drug/polymer compositions either in solution or in melt form to produce nanofibers.

FIG. 2 shows the X-Ray powder diffraction (XRPD) of electrospun 6-Acetyl-3,4-dihydro-2,2-dimethyl-trans(+)-4-(4-fluorobenzoylamino)-2H-benzo[b]pyran-3-ol hemihydrate fibers during storage up to 161 days at 25° C. Comparison with XRPD of the crystalline compound also shown in the figure, confirms the amorphous nature of the electrospun fiber.

FIG. 3 demonstrates the enhanced in vitro dissolution profiles of electrospun amorphous 6-Acetyl-3,4-dihydro-2,2-dimethyl-trans(+)-4-(4-fluorobenzoylamino-2H-benzo[b]pyran-3-ol hemihydrate fibers in comparison to crystalline ones.

FIG. 4 shows the XRPDs of electrospun 3-Hydroxy-2-phenyl-N-[1-phenylpropyl]-4-quinoline carboxamide (Talnetant) fibers during storage up to 120 days at 25° C., room temperature. For comparison XRPD of the crystalline drug and PVP are included in the figure. The X-ray difractograms show a halo, without any sharp peaks, attesting to the amorphous nature of the electrospun sample.

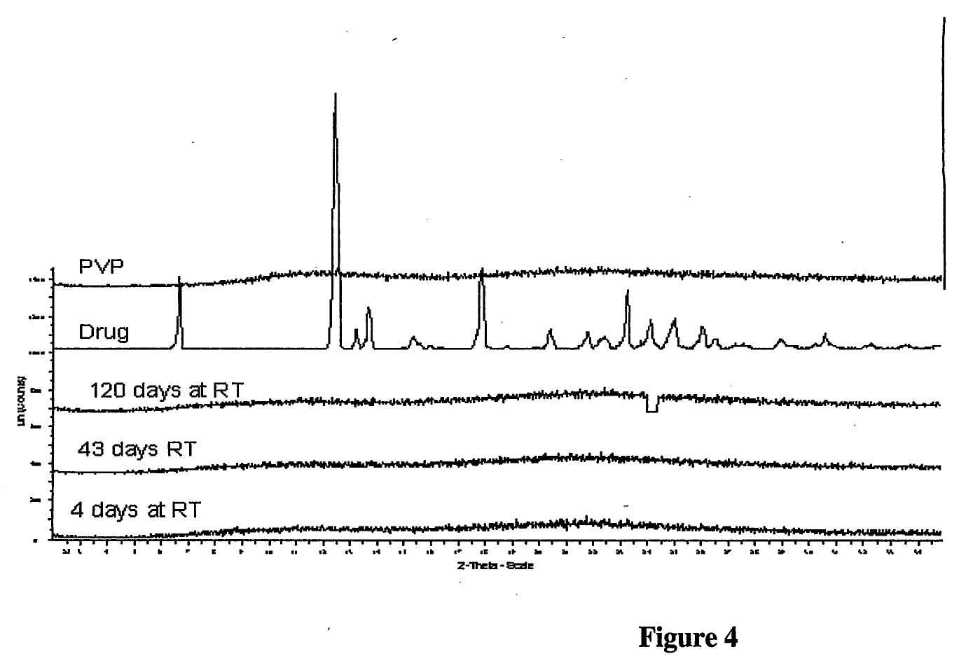

### DETAILED DESCRIPTION OF THE INVENTION

The present invention is directed to the discovery that the technique of electrospinning, i.e. the process of making polymer nanofibers from either a solution or melt under electrical forces, can be used to prepare stable, solid dispersions of an amorphous form of a drug in a polymer nanofibers.

Amorphous solids are disordered materials, which have no long-range order like crystalline materials. Amorphous materials exhibit both compositional and structural disorder. There is a distinguishing difference between compositional disorder and structural disorder. In compositional disorder, atoms are located in an ordered array like in crystalline materials. The spacing of the atoms is equidistant, but only the type of atom is placed randomly. In structural disorder, all bond distances have random lengths and random angles. Therefore there is no long range order, and hence no definite X-ray diffraction patterns. Amorphous solid is a glass in which atoms and molecules exist in a totally non-uniform array. Amorphous solids have no faces and cannot be identified as either habits or polymorphs. Because the properties of amorphous solids are direction independent, these solids are called isotropic. Amorphous solids are characterized by a unique glass transition temperature, the temperature at which it changes from a glass to rubber.

Due to the absence of long-range order, amorphous materials are in an unstable (excited state) equilibrium, resulting in physical as well as chemical instability. The physical instability manifests itself in higher intrinsic aqueous solubility compared to the crystalline drug. The higher solubility of the amorphous drug leads to a higher rate of dissolution, and to better oral bioavailability.

The pharmaceutical industry makes use of the amorphous state of a poorly soluble drug to enhance its aqueous solubility, and its oral bioavailability. However, as stated above, the amorphous state has undesirable physical and chemical instability. This can be overcome by blending the amorphous drug with appropriate polymers, to stabilize the amorphous state, for the desired shelf-life of the drug. It has been reported [Zografi et al, Pharm. Res. 1999, 16, 1722-1728] that the polymer-drug combination should have some specific interaction for stabilization of the amorphous drug.

The electrospun fibers of the present invention are expected to have diameters in the nanometer range, and hence provide a very large surface area. This extremely high surface area can dramatically increase the dissolution rate of the high molecular weight polymeric carrier as well as drug present in them.

A suitable dosage form, such as oral or parenteral forms, including pulmonary administration, may be designed by judicious consideration of polymeric carriers, in terms of their physio-chemical properties as well as their regulatory status. Other pharmaceutically acceptable excipients may be included to ameliorate the stabilization or de-agglomeration of the amorphous drug nanoparticles. The pharmaceutical excipients might also have other attributes, such as absorption enhancers.

Electrospun pharmaceutical dosage forms may be designed to provide any number of dissolution rate profiles, such as rapid dissolution, immediate, or delayed dissolution, or a modified dissolution profile, such as a sustained and/or pulsatile release characteristic.

Taste masking of the active agent may also be achieved by using polymers having functional groups capable of promoting specific interactions with the drug moiety. The electrospun dosage forms may be presented in conventional dosage formats, such as compressed tablets, capsules, sachets or films. These conventional dosage forms may be in the form of immediate, delayed and modified release systems, which can be designed by the appropriate choice of the polymeric carrier with the active agent/drug combination, using techniques well known and described in the art.

It is one embodiment of the present invention to provide drug particles in their amorphous form, embedded homogeneously in polymeric nanofibers, such that the drug is readily bioavailable independent of the route of administration.

It is another embodiment of the present invention to provide nanoparticle size drug particles having an amorphous morphology, which are embedded homogeneously within the polymeric nanofibers.

The starting compound as used herein, may be morphologically either in a crystalline state, or in an amorphous state. As can be seen herein, the present invention provides a novel vehicle which provides a means to allow a crystalline form of a drug to be stabilized in its amorphous form, or to take an amorphous form of a drug and retain its morphology in a controlled environment, i.e. the spun fibers. This can be used as noted, as a means to increase the surface area (nanoparticle size, etc.) and to improve its dissolution rate characteristics.

Electrospinning, commonly referred to as electrostatic spinning, is a process of producing fibers, with diameters in the range of 100 nm. The process consists of applying a high voltage to a polymer solution or melt to produce a polymer jet. As the jet travels in air, the jet is elongated under repulsive electrostatic force to produce nanofibers. The process has been described in the literature since the 1930. A variety of polymers both natural and synthetic having optimal characteristics have been electrospun under appropriate conditions to produce nanofibers, (see Reneker et al., Nanotechnology, 1996, 7, 216). Different applications have been suggested for these electrospun nanofibers, such as air filters, molecular composites, vascular grafts, and wound dressings.

U.S. Pat. No. 4,043,331, is intended for use as a wound dressing whereas U.S. Pat. No. 4,044,404, and U.S. Pat. No. 4,878,908 are tailored towards creating a blood compatible lining for a prosthetic device. All of the disclosed water insoluble polymers are not pharmaceutically acceptable for use herein, however the water soluble polymers disclosed are believed to be pharmaceutically acceptable. None of the preparations in these patents disclose a working example of an electrospun fiber with an active agent. The patents claim the use of enzymes, drugs and/or active carbon on the surface of the nanofibers, prepared by immobilizing the active moieties so that they act at the site of application and “do not percolate throughout the body”.

EP 542514, U.S. Pat. No. 5,311,884 and U.S. Pat. No. 5,522,879 pertain to use of spun fibers for a piezoelectric biomedical device. The piezoelectric properties of fluorinated polymers, such as those derived from a copolymer of vinylidene fluoride and tetrafluoroethylene are not considered pharmaceutically acceptable polymers for use herein.

U.S. Pat. No. 5,024,671 uses the electrospun porous fibers as a vascular graft material, which is filled with a drug in order to achieve a direct delivery of the drug to the suture site. The porous graft material is impregnated (not electrospun) with the drug and a biodegradable polymer is added to modulate the drug release. The vascular grafts are also made from non-pharmaceutically acceptable polymers, such as the polyterafluorethylene or blends thereof.

U.S. Pat. No. 5,376,116, U.S. Pat. No. 5,575,818, U.S. Pat. No. 5,632,772, U.S. Pat. No. 5,639,278 and U.S. Pat. No. 5,724,004 describe one form or another of a prosthetic device having a coating or lining of an electrospun non-pharmaceutically acceptable polymer. The electrospun outer layer is post-treated with a drug such as disclosed in the '116 patent (for breast prosthesis). The other patents describe the same technology and polymers but apply the technique to other applications, such as endoluminal grafts or endovascular stents.

Consequently, the present invention is the first to produce an electrospun composition of a pharmaceutically acceptable polymer in which one or more pharmaceutically acceptable active agents or drugs are stabilized in their amorphous form. The homogenous nature of this process produces a quantity of fibers which allow for nanoparticles of drugs to be dispersed throughout. The size of particle, and quality of dispersion provide for a high surface area of drug. One use of the increased surface area of drug is improved bioavailability in the case of a poorly water soluble drug. Other uses would be for decreased drug-drug or enzymatic interactions.

Yet another use of the present invention is to delay the release of drugs in the gastrointestinal tract by using pH sensitive polymers, such as the Eudgragit group of polymers by Rohm, in particular the Eudragit L100-55 polymer.

The present invention is therefore directed to use in any form of an electrospun drug/polymer combination, wherein the drug is stabilized in the amorphous form; and another wherein the resulting drug/polymer combination provides for enhanced bioavailability of the poorly soluble drug or to modify the absorption profile of the drug(s). The modification of the rate of release of the active compound when incorporated within the polymeric fibers may be increased or decreased. The resulting bioavailability of the active agent may also be increased or decreased relative to the immediate release dosage form.

While the application of this process may be of use for incorporation of a pharmaceutically acceptable drug for topical delivery, a preferred route of administration is likely to be oral, intravenous, intramuscular, or inhalation.

A pharmaceutically acceptable agent, active agent or drug as defined herein follows the guidelines from the European Union Guide to Good Manufacturing Practice: Any substance or mixture of substances intended to be used in the manufacture of a drug (medicinal) product and that, when used in the production of a drug, becomes an active ingredient of the drug product. Such substances are intended to furnish pharmacological activity or other direct effect in the diagnosis, cure, mitigation, treatment, or prevention of disease or to affect the structure and function of the body. Preferably, their use is in a mammal, more preferably a human. The pharmacological activity may be prophylactic or for treatment of a disease state. The pharmaceutical compositions described herein may optionally comprise one or more pharmaceutically acceptable active agents or ingredients distributed within.

As used herein the terms “agent”, “active agent”, “drug moiety” or “drug” are used interchangeably.

Water solubility of the active agent is defined by the United States Pharmacoepia.

Therefore, active agents which meet the criteria of very soluble, freely soluble, soluble and sparingly soluble as defined therein are encompassed this invention. It is believed that the electrospun polymeric composition, which most benefits those drugs, are those which are insoluble or sparingly soluble. However, as the electrospun polymeric composition produces, or stabilizes an amorphous form of the drug, the solubility of the drug may not be as important than if it were in a crystalline state.

The fibers of this invention will contain high molecular weight polymeric carriers. These polymers, by virtue of their high molecular weight, form viscous solutions that can produce nanofibers, when subjected to an electrostatic potential. The nano fibers spun electostatically may have a very small diameter. The diameter may be as small as 0.1 nanometers, more typically less than 1 micron. This provides a high surface area to mass ratio. The fiber may be of any length, and it may include particles which vary from the more traditional spun cylindrical shape such as drop-shaped or flat.

Suitable polymeric carriers can be preferably selected from known pharmaceutical excipients. The physico-chemical characteristics of these polymers dictate the design of the dosage form, such as rapid dissolve, immediate release, delayed release, modified release such as sustained release, or pulsatile release etc.

The delivery rate of the active agent can be controlled by varying the choice of the polymer used in the fibers, the concentration of the polymer used in the fiber, the diameter of the polymeric fiber, and/or the amount of the active agent loaded in the fiber.

Suitable drug substances can be selected from a variety of known classes of drugs including, for example, analgesics, anti-inflammatory agents, anthelmintics, anti-arrhythmic agents, antibiotics (including penicillins), anticoagulants, antidepressants, antidiabetic agents, antiepileptics or anticonvulsants (also referred to as neuroprotectants, antihistamines, antihypertensive agents, antimuscarinic agents, antimycobactefial agents, antineoplastic agents, immunosuppressants, antithyroid agents, antiviral agents, anxiolytic sedatives (hypnotics and neuroleptics), astringents, beta-adrenoceptor blocking agents, blood products and substitutes, cardiac inotropic agents, corticosteroids, cough suppressants (expectorants and mucolytics), diagnostic agents, diuretics, dopaminergics (antiparkinsonian agents), haemostatics, immunological agents, lipid regulating agents, muscle relaxants, NK3 receptor antagonists, parasympathomimetics, parathyroid calcitonin and biphosphonates, prostaglandins, radiopharmaceuticals, sex hormones (including steroids), anti-allergic agents, stimulants and anorexics, sympathomimetics, thyroid agents, PDE IV inhibitors, vasodilators and xanthines.

Preferred drug substances include those intended for oral administration and intravenous administration. A description of these classes of drugs and a listing of species within each class can be found, for example, in Martindale, The Extra Pharmacopoeia, Twenty-ninth Edition, The Pharmaceutical Press, London, 1989, the disclosure of which is hereby incorporated herein by reference in its entirety. The drug substances are commercially available and/or can be prepared by techniques known and described in the art.

As noted, the electrospun composition may also be able to taste mask the many bitter or unpleasant tasting drugs, regardless of their solubility. Suitable active ingredients for incorporation into fibers of the present invention include the many bitter or unpleasant tasting drugs including but not limited to the histamine H₂-antagonists, such as, cimetidine, ranitidine, famotidine, nizatidine, etinidine; lupitidine, nifenidine, niperotidine, roxatidine, sulfotidine, tuvatidine and zaltidine; antibiotics, such as penicillin, ampicillin, amoxycillin, and erythromycin; acetaminophen; aspirin; caffeine, dextromethorphan, diphenhydramine, bromopheniramine, chloropheniramine, theophylline, spironolactone, NSAIDS's such as ibuprofen, ketoprofen, naprosyn, and nabumetone; SHT₄ inhibitors, such as granisetron, or ondansetron; seratonin re-uptake inhibitors, such as paroxetine, fluoxetine, and sertraline; vitamins such as ascorbic acid, vitamin A, and vitamin D; dietary minerals and nutrients, such as calcium carbonate, calcium lactate, etc., or combinations thereof.

Suitably, the above noted active agents, in particular the anti-inflammatory agents, may also be combined with other active therapeutic agents, such as various steroids, decongestants, antihistamines, etc., as may be appropriate in either the electrospun fiber or in the resulting dosage form.

Preferably, the active agents are 6-Acetyl-3,4-dihydro-2,2-dimethyl-trans(+)-4-(4-fluorobenzoylamino)-2H-benzo[b]pyran-3-ol hemihydrate, 3-Hydroxy-2-phenyl-N-[1-phenylpropyl]-4-quinoline carboxamide (Talnetant), rosiglitazone, carvedilol, hydrochlorothiazide, eprosartan, indomethacin, nifedipine, naproxen, ASA, and ketoprofen, or those described in the Examples section herein.

The relative amount of fiber forming material (primarily the polymeric carrier) and the active agent that may be present in the resultant fiber may vary. In one embodiment the active agent comprises from about 1 to about 50% w/w of the fiber when electrospun, preferably from about 35 to about 45% w/w.

DNA fibers have also been used to form fibers by electrospinning, Fang et al., J. Macromol. Sci.-Phys., B36(2), 169-173 (1997). Incorporation of a pharmaceutically acceptable active agent, such as a biological agent, a vaccine, or a peptide, with DNA, RNA or derivatives, should they be amorphous, as a spun fiber is also within the scope of this invention.

The fiber forming characteristics of the polymer are exploited in the fabrication of nanofibers. Hence, molecular weight of the polymer is one of the single most important parameter for choice of polymer.

Another important criteria for polymer selection is the miscibility between the polymer and the drug. It may be theoretically possible to ascertain the miscibility's by comparing the solubility parameters of the drug and polymer, as described by Hancock et al, in International Journal of Pharmaceutics, 1997, 148, 1.

Another important criteria for polymer selection is its ability to stabilize the amorphous drug. It has been reported by Hancock et al, in Journal of Pharmaceutical Sciences, 1997, 86,1; that stable drug/polymer compositions should have glass transition temperatures (Tg) above the storage temperature. If the Tg of the drug/polymer combination is lower than the storage temperature, the drug will exist in the rubbery state, and will consequently be prone to molecular mobility and crystallisation. An example of this is the polymer poly(ethylene oxide) which is a semicrystalline/crystalline polymer. It has been shown that at least some crystalline drugs spun in such a polymer, having an amorphous morphology initially, will over time crystallize out.

Representative examples of amorphous polymers for use herein include, but are not limited to, polyvinyl alcohol, polyvinyl acetate, polyvinyl pyrrolidone, hyaluronic acid, alginates, carragenen, cellulose derivatives such as carboxymethyl cellulose sodium, methyl cellulose, ethylcellulose, hydroxyethyl cellulose, hydroxypropylcellulose, hydroxypropylmethyl cellulose, hydroxypropylmethyl cellulose phthalate, cellulose acetate phthalate, noncrystalline cellulose, starch and its derivatives such as hydroxyethyl starch, sodium starch glycolate, chitosan and its derivatives, albumen, gelatin, collagen, polyacrylates and methacrylic acid copolymers and their derivatives such as are found in the Eudragit family of polymers available from Rohm Pharma, poly(alpha-hydroxy acids) and its copolymers such poly(alpha-aminoacids) and its copolymers, poly(orthoesters), polyphosphazenes, polyethyloxazolines, poly(phosphoesters), and or combinations thereof.

The polymers, poly(ε-caprolactone), poly(oactide-co-glycolide), polyanhydrides, poly(ethylene oxide), are crystalline or semicrystalline polymers.

Most of these pharmaceutically acceptable polymers are described in detail in the Handbook of Pharmaceutical excipients, published jointly by the American Pharmaceutical association and the Pharmaceutical society of Britain.

Preferably, the polymeric carriers are divided into two categories, water soluble polymers useful for immediate release of the active agents, and water insoluble polymers useful for controlled release of the active agents. It is recognized that combinations of both carriers may be used herein. It is also recognized that several of the polyacrylates are pH dependent for the solubility and may fall into both categories.

Water soluble polymers include but are not limited to, polyvinyl alcohol, polyvinyl pyrrolidone, hyaluronic acid, alginates, carragenen, cellulose derivatives such as carboxymethyl cellulose sodium, hydroxyethyl cellulose, hydroxypropylcellulose, hydroxypropylmethyl cellulose, hydroxypropylmethyl cellulose phthalate, cellulose acetate phthalate, starch and its derivatives such as hydroxyethyl starch, sodium starch glycolate, dextrin, chitosan and its derivatives, albumen, zein, gelatin, and collagen.

A suitable water soluble polymer for use herein is polyvinylpyrrolidone, or polyvinylpyrrolidone and its copolymer with polyvinylacetate.

Water insoluble polymers include but are not limited to, polyvinyl acetate, methyl cellulose, ethylcellulose, noncrystalline cellulose, polyacrylates and its derivatives such as the Eudragit family of polymers available from Rohm Pharma (Germany), poly(alpha-hydroxy acids) and its copolymers such as poly(alpha-aminoacids) and its copolymers, poly(orthoesters), polyphosphazenes, and poly(phosphoesters).

The acrylic polymers of the Eudragit family are well known in the art and include a number of different polymers, ranging from Eudragit L100-55 (the spray dried form of Eudragit L30D), L30D, L100, S 100, 4135F, E100, EPO (powder form of E100), RL30D, RL PO, RL 100, RS 30D, RS PO, RS 100, NE 30 D, and NE 40 D.

These pharmaceutically acceptable polymers and their derivatives are commercially available and/or be prepared by techniques known in the art. By derivatives it is meant, polymers of varying molecular weight, modification of functional groups of the polymers, or co-polymers of these agents, or mixtures thereof.

Further, two or more polymers can be used in combination to form the fibers as noted herein. Such combination may enhance fiber formation or achieve a desired drug release profile. One suitable combinations of polymers includes polyethyleoxide and polycaprolactone.

Preferably, the polymer of choice is an amphorous polymer, such as but not limited to: polyvinyl alcohol, polyvinyl acetate, polyvinyl pyrrolidone, hyaluronic acid, alginates, carragenen, cellulose derivatives such as carboxymethyl cellulose sodium, methyl cellulose, ethylcellulose, hydroxyethyl cellulose, hydroxypropylcellulose, hydroxypropylmethyl cellulose, hydroxypropylmethyl cellulose phthalate, cellulose acetate phthalate, noncrystalline cellulose, starch and its derivatives such as hydroxyethyl starch, sodium starch glycolate, chitosan and its derivatives, albumen, gelatin, collagen, polyacrylates and its derivatives such as the Eudragit family of polymers available from Rohm Phanna, such as Eudragit L100-55, poly(alpha-hydroxy acids), poly(alpha-aminoacids) and its copolymers, poly(orthoesters), polyphosphazenes, and poly(phosphoesters). The preferred polymers are ones with functional groups capable of promoting specific interaction with the active agent to help stabilize the amorphous form of the agent. Suitable polymers are PVP and PVP with copolymers or the Eudgragit group of polymers as described herein.

The choice of polymers taken with the active agent may provide suitable taste masking functions for the active agents. For instance, use of an ionic polymer of contrasting charge, such as a cationic polymer complexed with an anionic active agent, or an anionic polymer complexed with a cationic active agent may produce the desired results. Addition of a second taste masking agent, such as a suitable cyclodextrin, or its derivatives may also be used herein.

The polymeric composition may be electrospun from a solvent base or neat (as a melt). Solvent choice is preferably based upon the solubility of the active agent. Suitably, water is the best solvent for a water soluble active agent, and polymer. Alternatively, water and a water miscible organic solvent may used. However, it is necessary to use an organic solvent to prepare a homogenous solution of the drug with polymer when the drug is non-water soluble, or sparingly soluble.

It is recognized that these polymeric compositions which are spun neat may also contain additional additives such as, plasticizers, and antioxidants. The plasticizers are employed to assist in the melting characteristics of the composition. Exemplary of plasticizers that may be employed in the coatings of this invention are triethyl citrate, triacetin, tributyl citrate, acetyl triethyl citrate, acetyl tributyl citrate, dibutyl phthalate, dibutyl sebacate, vinyl pyrrolidone and propylene glycol.

Preferably, tie solvent of choice is a GRASS approved organic solvent, although the solvent may not necessarily be “pharmaceutically acceptable” one, as the resulting amounts may fall below detectable, or set limits for human consumption they may be used. It is suggested that ICH guidelines be used for selection.

Suitable solvents for use herein include, but are not limited to acetic acid, acetone, acetonitrile, methanol, ethanol, propanol, ethyl acetate, propyl acetate, butyl acetate, butanol, N,N dimethyl acetamide, N,N dimethyl formamide, 1-methyl-2-pyrrolidone, dimethyl sulfoxide, diethyl ether, dilsopropyl ether, tetrahydrofuran, pentane, hexane, 2-methoxyethanol, formamide, formic acid, hexane, heptane, ethylene glycol, dioxane, 2-ethoxyethanol, trifluoroacetic acid, methyl isopropyl ketone, methyl ethyl ketone, dimethoxy propane, methylene chloride etc., or mixtures thereof.

A preferred solvent is ethanol, acetone, n-vinylpyrrolidone, dichloromethane, acetonitrile, tetrahydrofuran or a mixture of these solvents.

The solvent to polymeric composition ratio is suitable determined by the desired viscosity of the resulting formulation.

For electrospinning of a pharmaceutical polymeric composition, key parameters are viscosity, surface tension, and electrical conductivity of the solvent/polymeric composition.

By the term “nanoparticulate drug” as used herein, is meant, nanoparticule size of an active agent within the electrospun fiber, as opposed to a nanoparticule size of the resulting fibers themselves.

The polymeric carriers may also act as surface modifiers for the nanoparticulate drug. Therefore, a second oligomeric surface modifier may also be added to the electrospinning solution. All of these surface modifiers may physically adsorb to the surface of the drug nanoparticles, so as to prevent them agglomerating.

Representative examples of these second oligomeric surface modifier or excipients, include but are not limited to: Pluronics® (block copolymers of ethylene oxide and propylene oxide), lecithin, Aerosol OT™ (sodium dioctyl sulfosuccinate), sodium lauryl sulfate, Tween™, such as Tween 20, 60 & 80, Span™, Arlacel™, Triton X-200, polyethylene glycols, glyceryl monostearate, Vitamin E-TPGS™ (d-alpha-tocopheryl polyethylene glycol 1000 succinate), sucrose fatty acid esters, such as sucrose stearate, sucrose oleate, sucrose palmitate, sucrose laurate, and sucrose acetate butyrate etc.

Triton X-200 is Polyethylene glycol octylphenyl ether sulfate ester sodium salt; or Polyethylene glycol octylphenyl ether sulfate sodium salt. Span and Arlacel are synonyms for a sorbitan fatty acid ester as defined in the Handbook of Pharmaceutical Excipients, and Tween is also a synonym for polyoxyethylene sorbitan fatty acid esters.

Surfactants are added on a weight/weight basis to the drug composition. Suitably, the surfactants are added in amounts of up to 15%, preferably about 10%, preferably about 5% or less. Surfactants can lower the viscosity and surface tension of the formulation, and in higher amounts can adversely effect the quality of the electrospun fibers.

The surfactant selection may be guided by HLB values but is not necessarily a useful criteria. While HLB surfactants have been utilised herein, such as Tween™ 80 (HLB=10), Pluronic F68 (HLB=28), and SDS (HLB>40), lower HLB value surfactants, such as Pluronic F92 may also be used.

Another pharmaceutically acceptable excipients may be added to the electrospinning composition. These excipients may be generally classified as absorption enhancers, flavouring agents, dyes, etc.

The polymeric carriers or the second oligomeric surface modifiers, if appropriately chosen, may themselves act as absorption enhancers, depending on the drug. Suitable absorption enhancers for use herein, include but are not limited to, chitosan, lecithin, lectins, sucrose fatty acid esters such as the ones derived from stearic acid, oleic acid, palmitic acid, lauric acid, and Vitamin E-TPGS, and the polyoxyethylene sorbitan fatty acid esters.

Use of the electrospun composition herein may be by conventional capsule or tablet fill as well known in the art. Alternatively, the fibers may be ground, suitably by cryogenic means, for compression into a tablet or capsule, for use by inhalation, or parenteral administration. The fibers may also be dispersed into an aqueous solution, which may then be directly administered by inhaled or given orally. The fibers may also be cut, optionally milled, and processed as a sheet for further administration with agents to form a polymeric film, which may be quick-dissolving.

An alternative electrospinning process for making the pharmaceutical compositions described herein is also possible. The Examples herein electrostatically charge the solution whereas the pharmaceutical composition may also be ejected from a sprayer onto a receiving surface that is electrostatically charged and placed at an appropriate distance from the sprayer. As jet travels in air from the sprayer towards the charged collector, fibers are formed. The collectors can be either a metal screen, or in the form of a moving belt. The fibers deposited on the moving belt are continuously removed and taken away.

### EXAMPLES

General Procedure for Electrospinning

A solution of the drug and polymer in a suitable organic solvent is electrospun using the following electrospinning set up. The solution to be electrospun is taken in a 25 ml glass vessel having a 0.02 mm capillary outlet at the bottom and two top inlets, one for applying a positive He pressure and the other for introducing the electrode through a rubber septum. The electrode is connected to the positive terminal of a high voltage power supply (Model ES30P/M692, Gamma High Voltage Research Inc., FL). The ground from the high voltage power supply is connected to a stainless steel rotating drum, which acts the collector for the fibers. A voltage of 18-25 KV is applied to the polymer solution through the electrode which reaches the bottom of the glass vessel. This high voltage creates a monofilament from the capillary outlet and the monofilament is further splayed to form nanofibers. The inlet He pressure varying from 0.5-2 psi is adjusted to maintain a constant feed of liquid to the capillary tip, in order to produce continuous electrospinning and to prevent the formation of excess liquid droplets, which might simply fall off from the capillary. The rotating drum is kept a distance of 15-25 cm from the positive electrode. The dry fibers collected on the drum is peeled off and harvested.

Materials

Polyvinylpyrrolidone (PVP), molecular weight 1.3M, available from Sigma-Aldrich Chemicals (St.Louis, Mo.) and polyvinylpyrrolidone-co-polyvinylacetate (Kolloidon VA-64), available from BASF, Eudragit L100 55 (Rohm Pharma), polyethylene oxide as POLYOX WSR 1105 (Union Carbide) are used for experiments. Drug substances such as, rosiglitazone, carvedilol, eprosartan, hydrochlorothiazide, indomethacin, nifedipine, ketoprofen, and naproxen are available commercially from the manufacturer or from various catalogs, such as Sigma-Aldrich.

Methods

Drug Content

Drug content in the electrospun samples were determined by an appropriate HPLC method. A weighed amount of electrospun fibers, is dissolved in a solvent and analyzed by Agilent 1100 HPLC system having a C18 column.

In Vitro Dissolution Assay

The equipment used for this procedure is a modified USP 4, the major differences being: 1. low volume cell. 2. stirred cell. 3. retaining filters which are adequate at retaining sub micron material. The total run time is 40 minutes. 2.5 mg of drug (weigh proportionally more formulated material).

Flow Cell Description: Swinnex filter assemblies obtained from Millipore, having 0.2 micron Cellulose Nitrate membranes. (Millipore, Mass.) as internal filters. The internal volume of the cell is approximately 2 ml. A Small PTFE stirrer customized to fit the Swinnex assembly (Radleys Lab Equipment Halfround Spinvane F37136) is used. The dissolution medium at a flow rate of 5 ml/min is used. The whole set up is placed at a thermostat of 37° C. The drug concentration is measured by passing the eluent through a UV detector having a flow cell dimension of 10 mm. The UV detection is carried out at an appropriate wavelength for the drug.

Determination of Extent of Drug Solubility

The experimentation-is designed to evaluate drug dissolution rate. As such it is unlikely with poorly soluble drugs, and with water as the dissolution medium, that 100% of the drug will dissolve in the 40 minute duration of the test. To determine the extent of drug solubility over this period one collects all 200 ml of solution that elutes from the dissolution cell. Using a conventional UV spectrophotometer, this solution is compared against a reference solution of 2.5 or 4 mg of active agent dissolved in a suitable medium.

Amorphicity and its Stability Over Time

The am orphous nature of the drug in the formulation and its stability on ageing at 25° C. and zero humidity, was determined by XRPD. The instrument is a Bruker D8 AXS Diffractometer. Approximately 30 mg of sample is gently flattened on a silicon sample holder and scanned at from 2-35 degrees two-theta, at 0.02 degrees two-theta per step and a step time of 2.5 seconds. The sample is rotated at 25 rpm to reduce preferred orientation. Generator power is set at 40 mA and 40 kV.

The amorphous nature of the drug was also confirmed by MDSC (TA instruments, New Castle, Del.). The samples in hermetically sealed aluminium pans were heated from 0 to 200, or to 250° C. at 2° C./min at a modulation frequency of ±0.159° C. every 30 seconds.

### Example 1

### Preparation of amorphous 6-Acetyl-3,4-dihydro-2,2-dimethyl-trans(+)-4-(4-fluorobenzoylamino)-2H-benzo[b]pyran-3-ol hemihydrate (Compound I) by electrospinning

Various samples shown in Table 1, were prepared by dissolving the title compound and PVP in ethanol. This solution was electrospun using the set up described in the experimental section above.

TABLE 1

| Ingredients | Sample 1.1 | | Sample 1.2 | | Sample 1.3 | |
|---|---|---|---|---|---|---|
| Compound I | 300 | mg | 400 | mg | 2 | g |
| PVP | 600 | mg | 600 | mg | 3 | g |
| Ethanol used | 10 | ml | 7 | ml | 40 | ml |

| | | | |
|---|---|---|---|
| Surfactant (Tween 80) | 50 | mg | none |

| | | | | | |
|---|---|---|---|---|---|
| Yield (g) | 400 | mg | n/a | 4 | g |

| | | | |
|---|---|---|---|
| Drug content | 37.3% | 37.1% | 33.3% |
| determined by HPLC | | | |

XRPD of the Electrospun Compound I, Sample 1.2

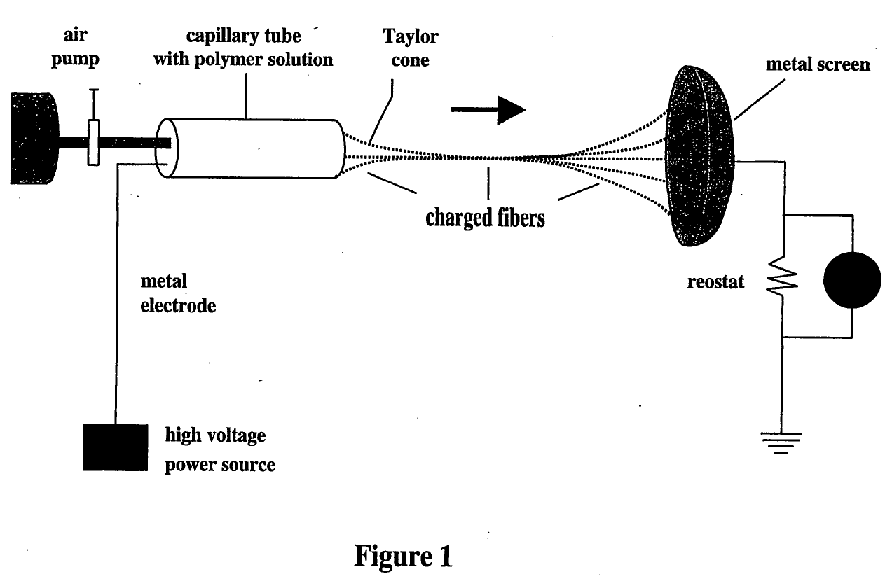

XRPDs of the electrospun sample 1.2 after storage at 25° C. and zero humidity for several days up to 161 days, show the sample to be amorphous. FIG. 1 compares the XRPDs of sample 1.2 stored for 45, 84, 133 and 161 days, along the XRPD of crystalline drug and PVP.

Thermal Analysis of Samples 1.2 and 1.3

Crystalline Compound I exhibits crystalline melting endotherm at 145° C., whereas the sample 1.2 and sample 1.3 do not have a crystalline melting endotherm, when heated from 0 to 200° C.

In Vitro Dissolution Rates

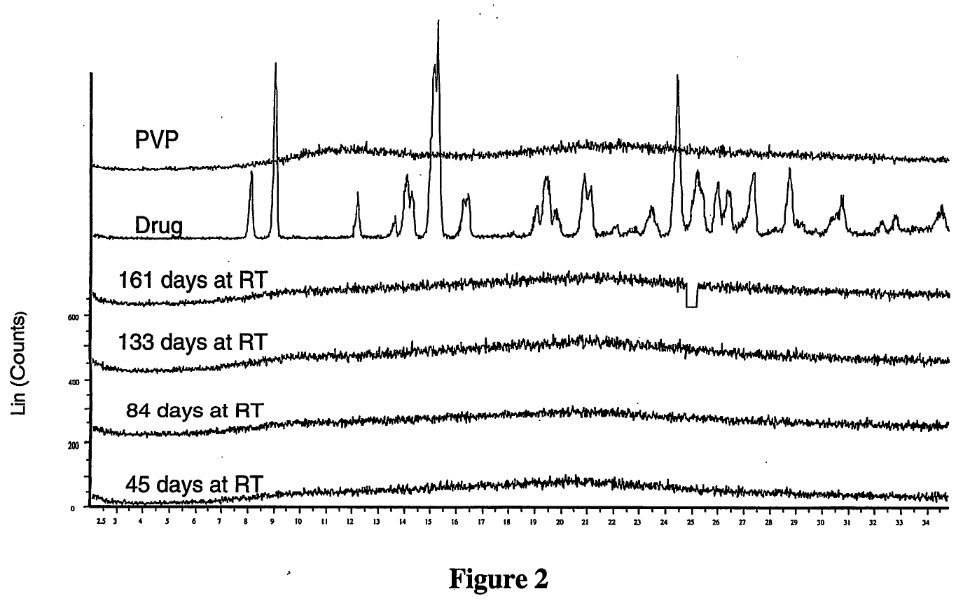

In vitro dissolution rates of samples 1.1, 1.2 and 1.3 were determined using the protocol described in the experimental section. The dissolution medium was a mixture of water and acetonitrile (8:2), and the wavelength used for drug detection 275 nm. Two different lots of unmilled Compound I were also used for comparison. The data shown in FIG. 2, indicates that the electrospun fibers have much higher dissolution rates than the crystalline drug.

The percentage drug dissolved at various time points are collated in the following table.

TABLE 2

| | | % Drug Dissolved | | | |
|---|---|---|---|---|---|
| Sample | Drug Content | 10 min | 20 min | 30 min | 40 min |
| Compound I | 99.5% | 17.4 | 24.3 | 29.4 | 33.8 |
| Compound I | | 12.1 | 18.2 | 23.2 | 27.8 |
| Sample 1.1 | 37.3 | 61.1 | 73.5 | 82 | 87.1 |
| Sample 1.2 | 37.1 | 52.4 | 67.7 | 78.5 | 84.1 |
| Sample 1.3 | 33.1 | 36.7 | 61.5 | 73.7 | 82 |

### Example 2

### Preparation of Amorphous Talnetant (Compound II) by Electrospinning

Talnetant HCl, (3-Hydroxy-2-phenyl-N-[(1S)-1-phenylpropyl]-4-quinolinecarboxamide monohydrochloride, also referred to as Compound II, is dissolved in a minimum amount of tetrahydrofuran (THF), and then requisite quantity of PVP and ethanol are added to form a clear yellow solution. This solution is electrospun using the set up. The fibers collected are yellowish in color. Different samples prepared are described in the following table.

| | Sample | Sample | Sample | Sample | Sample | Sample | Sample | Sample | Sample |
|---|---|---|---|---|---|---|---|---|---|
| Ingredients | 21 | 22 | 23 | 24 | 25 | 26 | 27 | 28 | 29 |
| Compnd II | 400 mg | 400 | 400 | 2 g | 1 g | 2 g | 400 mg | 600 mg | 600 mg |
| THF | 2 ml | 2 ml | 2 ml | 5 ml | 2.5 ml | 5 ml | 1.4 ml | 2.1 ml | 2.1 ml |
| PVP | 600 mg | 550 mg | 550 | 3 g | none | none | 550 mg | 860 mg | 860 mg |
| Kolloidon | none | none | none | none | 1.5 g | 3 g | none | none | none |
| VA 64 | | | | | | | | | |
| Ethanol | 10 ml | 10 ml | 10 ml | 50 ml | 10 ml | 20 ml | 10 ml | 13 ml | 13 ml |
| Surfactant | none | Tween | TPGS/ | none | none | none | Tween | none | none |
| | | 80/50 | 50 mg | | | | 80/50 | | |
| | | mg | | | | | mg | | |
| Yield | 900 mg | 850 mg | 860 mg | 3.8 g | 2.3 g | 4.4 g | 720 mg | 1065 mg | 1065 mg |
| Drug | 36.7% | 36.6% | 39.9% | 40.7% | 40.0% | 39.1% | 39.2% | 41.1% | 38.7% |
| content by | | | | | | | | | |
| HPLC | | | | | | | | | |

XRPD of the Electrospun Compound II, Sample 2.1

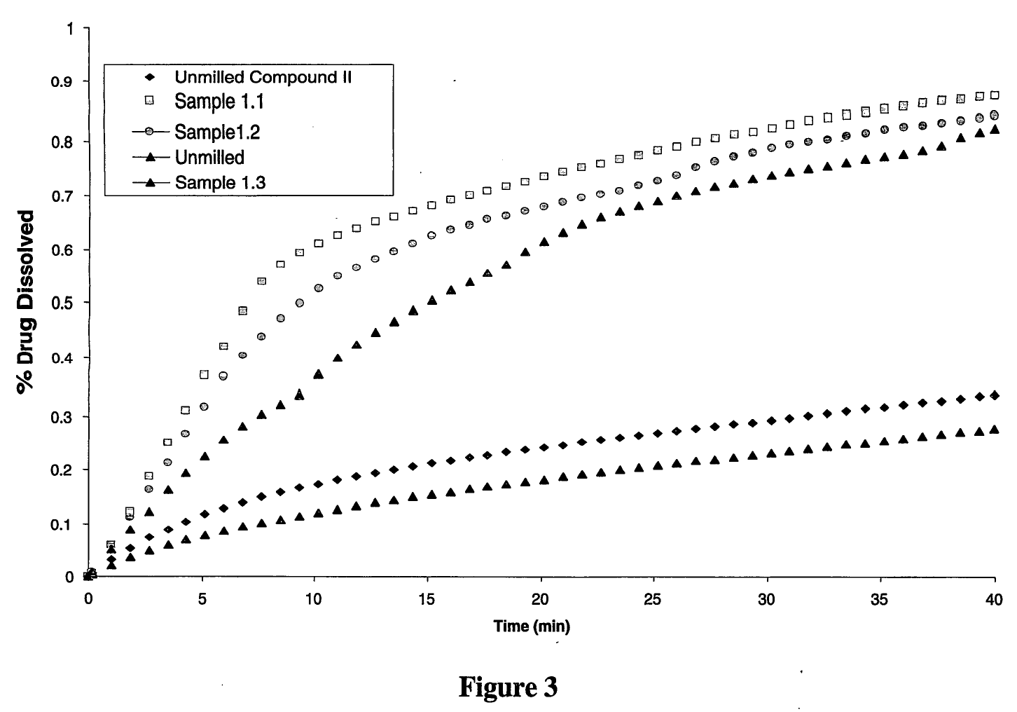

XRPDs of the electrospun sample 2.1 after storage at 25° C. and zero humidity for several days up to 161 days, show the sample to be amorphous. FIG. 3 compares the XRPDs of sample 1.2 stored for 4, 43, and 120 days, along the XRPD of crystalline drug and PVP.

Thermal Analysis of Samples 2.1, 2.2, 2.3, and 2.4

Crystalline Compound II exhibits crystalline melting endotherm at 161° C., whereas the electrospun samples 2.1, 2.2, 2.3 and 2.4 do not have a crystalline melting endotherm, when heated from 0 to 200° C.

MDSC Analysis of Sample 2.7 and 2.8

Analysis confirmed the drug to be in an amorphous state.

In Vitro Dissolution Rates

In vitro dissolution rates of samples 2.1, 2.2, 2.3, 2.4, 2.5 and 2.6 were determined using the protocol described in the experimental section. The dissolution medium was 0.1M HCl, and the wavelength used for drug detection 244 nm. An unmilled lot of Compound II was used for comparison. As shown in the Table below, the electrospun formulations have much faster rate of dissolution.

| | | % Drug Dissolved | | | |
|---|---|---|---|---|---|
| Sample | Drug Content | 10 min | 20 min | 30 min | 40 min |
| Compound II | 99.5% | 3.8 | 6.3 | 8.5 | 10.7 |
| Sample 2.1 | 36.7 | 15.7 | 30.1 | 43.8 | 59.1 |
| Sample 2.2 | 36.6 | 24.8 | 42.6 | 58.8 | 69.9 |
| Sample 2.3 | 39.9 | 19.6 | 44.9 | 62.8 | 75.9 |
| Sample 2.4 | 40.7 | 8.5 | 15.1 | 21.1 | 29.8 |
| Sample 2.5 | 40. | 19.8 | 31.1 | 41.1 | 50.1 |
| Sample 2.6 | 39.1 | 26.2 | 40.2 | 52.0 | 60.3 |

### Example 3

### Preparation of Amorphous Formulations of Various Drugs

Various drugs such as avandia, eprosartan, carvedilol, hydrochloridethiazide, aspirin, naproxen, nifedipine, indomethacin, and ketoprofen were solubilized in appropriate solvents and mixed with PVP dissolved in ethanol to form clear solutions. These solutions were electrospun using the set up described in the experimental section above, and fibers containing the amorphous drug were collected. The following table describes the various formulations used to prepare the electrospun samples.

TABLE 3

| | Amount | | | | | Amorphous | |
|---|---|---|---|---|---|---|---|
| Drug | of drug | Solvent | PVP | Ethanol | Yield | DSC | XRPD |
| Rosiglitazone | 350 mg | THF/8 ml | 550 mg | none | poor | yes | yes |
| Rosiglitazone | 350 mg | DCM*/ | 550 mg | 9 ml | poor | yes | yes |
| | | 3 ml | | | | | |
| Carvedilol | 700 mg | NMP**/ | 1.2 g | 6 ml | 0.3 g | yes | yes |
| | | 4 ml | | | | | |
| Eprosartan | 350 mg | NMP/3 ml | 600 mg | 6 ml | 0.2 g | yes | yes |
| Hydrochloro- | 400 mg | Acetone/ | 600 mg | 5 ml | 0.7 g | yes | yes |
| thiazide | | 3 ml | | | | | |
| Aspirin | 800 mg | Ethanol/ | 1.2 g | 5 ml | 1.8 g | yes | yes |
| | | 10 ml | | | | | |
| Naproxen | 800 mg | Ethanol/ | 1.2 g | 5 ml | 1.8 g | yes | yes |
| | | 10 ml | | | | | |
| Nifedipine | 800 mg | Ethanol/ | 1.2 g | 5 ml | 2 g | yes | yes |
| | | 10 ml | | | | | |
| Indomethacin | 800 mg | Aceto- | 1.2 g | 10 ml | 1.8 g | yes | yes |
| | | nitrile/ | | | | | |
| | | 5 ml | | | | | |

*DCM—Dichioromethane
*NMP—N-methyl pyrrolidone

### Example 4

### Electrospinning of 35.52% (w/w)Carvedilol HBr Monohydrate Composition

400 mg of crystalline material, Carvedilol HBr monohydrate was dissolved in 4.0 mL of tetrahydrofuran (Mallinckrodt) and 3 mL of MilliQ™ water. The drug solution was added to 600 mg of POLYOX WSR 1105 (Union Carbide) in 10 mL of acetonitrile (EM). The contents were mixed to form a solution. This polymer solution has 1441 μS/cm of conductivity and 676 Cp of viscosity. This solution was spun using similar conditions as described above in Example 4 above to yield 402 mg of nanofibers containing the title compound. The morphology of the drug using MDSC was confirmed as amorphous. Over time, the morphology of the drug will convert to a crystalline form.

### Example 5

### Electrospinning of (3R,3aS,6aR)-hexahydrofuro[2,3-b]furan-3-yl (1S,2R)-3-[(1,3-benzodioxol-5-ylsulfonyl)(isobutyl)amino]-2-hydroxy-1-{4-[(2-methyl-1,3-thiazol-4-yl)methoxy]benzyl}propylcarbamate-39.76% (w/w) composition

400 mg of the free base, crystalline form title compound was dissolved in 2.0 mL of methylene chloride (EM) The drug solution was added to 600mg of Eudragit L100-55 (Rohm) in 2.0 mL of ethanol (AAPER). This solution was spun using similar conditions as described above in Example 2, above to yield 340 mg of nanofibers containing the compound. The morphology of the drug using MDSC was confirmed as amorphous.

### Example 6

### Electrospinning of 37.58% (w/w) (3R,3aS,6aR)-hexahydrofuro[2,3-b]furan-3-yl(1S,2R)-3-[(1,3-benzodioxol-5-ylsulfonyl)(isobutyl)amino]-2-hydroxy-1-{4-[(2-methyl-1,3-thiazol-4-yl)methoxy]benzyl}propylcarbamate composition

500 mg of the title compound (crystalline form, free base) was dissolved in 2.5 mL of methylene chloride (EM) The drug solution was added to 700 mg of POLYOX WSR 1105 (Union Carbide) in 15 mL of acetonitrile (EM). 50 mg of Tween 80 (J. T. Baker) was added and polymer solution was clear. This solution was electrospun using similar conditions as described above in Example 2, above, to yield 774 mg of nanofibers containing the title compound. The morphology of the drug using MDSC and X-Ray diffraction was confirmed as crystalline.

Repeat synthesis of the fibers using the conditions set forth in his example yielded a drug load of 39.12% w/w, and 38.06%, respectively and the morphology determination by MDSC, and ID as crystalline.

### Example 7

### Electrospinning of 30.22% (w/w) (3R,3aS,6aR)-hexahydrofuro[2,3-b]furan-3-yl(1S,2R)-3-[(1,3-benzodioxol-5-ylsulfonyl)(isobutyl)amino]-2-hydroxy-1-{4-[(2-methyl-1,3-thiazol-4-yl)methoxy]benzyl}propylcarbamate composition

400 mg of the title compound (76.46%, tosylate salt) as an amorphous form, was dissolved in 3.0 mL of methylene chloride (EM) The drug solution was added to 600 mg of Eudragit L100-55 (Rohm) in 3.0 mL of ethanol (AAPER). 10 mg of Tween 80 (J. T. Baker) was added to the solution. This solution was electrospun using similar conditions as described above in Example 2, above, to yield 224 mg of nanofibers containing the compound. The morphology of the drug in the spun fiber using MDSC and X-Ray diffraction was confirmed as amorphous.

A repeat of this experiment yielded a drug content of 29.66% w/w and confirmed morphology using MDSC and X-Ray diffraction as amorphous.

### Example 8

### Electrospinning of 29.66% (w/w) (−)-(S)—N-[α-Ethylbenzyl)-3-hydroxy-2-phenyl quinoline-4-carboxamide HCl composition

600 mg of the title compound was dissolved in 2.1 mL of tetrahydrofuran (Aldrich). The drug solution was added to 1030 mg of POLYOX WSR 1105 (Union Carbide) in 26 mL of acetonitrile (EM) together with 80 mg of Tween 80 (J. T. Baker). The contents were mixed to form a solution, then the polymer solution was sonicated for fifteen minutes. The solution was electrospun using similar conditions as described above in Example 2, above to yield 636 mg of nanofibers containing the title compound. The morphology of the drug using MDSC and X-ray Diffraction was confirmed as crystalline.

### Example 9

### Electrospinning of 29.86% (w/w) (3R,3aS,6aR)-hexahydrofuro[2,3-b]furan-3-yl(1S,2R)-3-[(1,3-benzodioxol-5-ylsulfonyl)(isobutyl)amino]-2-hydroxy-1-}4-[(2-methyl-1,3-thiazol-4-yl)methoxy]benzyl}propylcarbamate (Tosylate) composition

400 mg of the title compound as the amorphous form, tosylate salt (strength 78.74%) was dissolved in 2.0 mL of methylene chloride (EM). The drug solution was added to 600 mg of POLYOX WSR 1105 (Union Carbide) in 23 mL of acetonitrile (EM) together with 60 mg of Tween 80 (J. T. Baker). The contents were mixed to form a solution. The solution was electrospun using similar conditions as described above in Example 2 above, to yield 339 mg of nanofibers containing the compound. The morphology of the drug using MDSC and X-Ray diffraction was confirmed as amorphous.

### Example 10

### Electrospinning of 29.08% (w/w) (3R,3aS,6aR)-hexahydrofuro[2,3-b]furan-3-yl(1S,2R)-3-[(1,3-benzodioxol-5-ylsulfonyl)(isobutyl)amino]-2-hydroxy-1-{4-[(2-methyl-1,3-thiazol-4-yl)methoxy]benzyl}propylcarbamate composition

800 mg of the title compound (crystalline form) was completely dissolved in 5.0 mL of methylene chloride (EM). 1300 mg of polycaprolactone(hereinafter “PCL”) and 400 mg of POLYOX WSR 1105 (Union Carbide) were added into drug solution together with 1 mL of acetonitrile (EM). The contents were mixed to form a solution. The solution was electrospun using similar conditions as described above in Example 2, above. 757 mg of nanofibers containing the compound were collected. The morphology of the drug substance as determined by MDSC was crystalline.

### Example 11

### Electrospinning of 48.46% (w/w) (3R,3aS,6aR)-hexahydrofuro[2,3-b]furan-3-yl(1S,2R)-3-[(1,3-benzodioxol-5-ylsulfonyl)(isobutyl)amino]-2-hydroxy-1-{4-[(2-methyl-1,3-thiazol-4-yl)methoxy]benzyl}propylcarbamatecomposition

800 mg of the title compound (crystalline form) was completely dissolved in 5.0 mL of methylene chloride (EM). 800 mg of PCL was added into drug solution together with additional 3.0 mL of methylene chloride (EM). The contents were mixed to form a solution. The solution was electrospun using similar conditions as described above in Example 2, above. 482 mg of nanofibers containing the compound were collected from the drum. The morphology of the drug substance as determined by MDSC was crystalline.

### Example 12

### Electrospinning of 39.14% (w/w) (3R,3aS,6aR)-hexahydrofuro[2,3-b]furan-3-yl(1S,2R)-3-[(1,3-benzodioxol-5-ylsulfonyl)(isobutyl)amino]-2-hydroxy-1-{4-[(2-methyl-1,3-thiazol-4-yl)methoxy]benzyl}propylcarbamate (Tosylate) composition

1000 mg of the title compound (amorphous form) was completely dissolved in 3.0 mL of methylene chloride (EM). The drug solution was added into 500 mg of PCL and 500 mg of POLYOX WSR 1105 (Union Carbide) in 13 mL of acetonitrile (EM) The resultant solution was electrospun using conditions similar to Example 2 above, but using a feed pressure of 1 psi. 1.5524 g of fibers were collected and removed from the drum. The morphology of the drug substance as determined by MDSC was amorphous.

### Example 13

### Electrospinning of 38.35% (w/w) (3R,3aS,6aR)-hexahydrofuro[2,3-b]furan-3-yl(1S,2R)-3-[(1,3-benzodioxol-5-ylsulfonyl)(isobutyl)amino]-2-hydroxy-1-{4-[(2-methyl-1,3-thiazol-4-yl)methoxy]benzyl}propylcarbamate composition

3.0 g of the free base, crystalline form title compound was dissolved in 15.0 mL of methylene chloride (EM) The drug solution was added to 4.5 g of Eudragit L100-55 (Rohm) in 22.0 mL of ethanol (AAPER). After that 98 mg of Tween 80 (J. T. Baker) was added into the polymer solution. This solution was spun using similar conditions as described above in Example 2, above to yield 5.2 g of nanofibers containing the compound. The morphology of the drug substance as determined by MDSC was amorphous.

### Example 14

### Electrospinning of ˜40% (w/w) 3-methyl-N-[(1S)-3-methyl-1-({[(4S,7R)-7-methyl-3-oxo-1-(2-pyridinylsulfonyl)hexahydro-1H-azepin-4-yl]amino}carbonyl)butyl]furo[3,2-b]pyridine-2-carboxamide composition

400 mg of the title compound, as an amorphous material was dissolved in 1.8 mL of tetrahydrofuran (Aldrich). The drug solution was added to 600 mg of POLY OX WSR 1105 (Union Carbide) in 16 mL of acetonitrile (EM). This solution was electrospun using similar conditions as described above in Example 2, to yield 85 mg of nanofibers containing the title compound. The morphology of the drug substance as determined by MDSC was amorphous.

All publications, including but not limited to patents and patent applications, cited in this specification are herein incorporated by reference as if each individual publication were specifically and individually indicated to be incorporated by reference herein as though fully set forth.

The above description fully discloses the invention including preferred embodiments thereof. Modifications and improvements of the embodiments specifically disclosed herein are within the scope of the following claims. Without further elaboration, it is believed that one skilled in the are can, using the preceding description, utilize the present invention to its fullest extent. Therefore, the Examples herein are to be construed as merely illustrative and not a limitation of the scope of the present invention in any way. The embodiments of the invention in which an exclusive property or privilege is claimed are defined as follows.

1. A pharmaceutical composition comprising an electrospun fiber of a pharmaceutically acceptable polymeric carrier homogeneously integrated with a stable amorphous form of a pharmaceutically acceptable active agent.

2. The composition according to claim 1 wherein the polymeric carrier is an amorphous polymer.

3. The composition according to claim 1 wherein the active agent is nanoparticle in size.

4. The composition according to claim 1 wherein the active agent is water soluble.

5. The composition according to claim 1 wherein the active agent is water insoluble.

6. The composition according to claim 1 wherein the active agent is sparingly water soluble.

7. The composition according to claim 1 wherein the polymeric carrier is water soluble.

8. The composition according to claim 1 wherein the polymeric carrier is water insoluble.

9. The composition according to claim 1 wherein the composition further comprises a surfactant which is a block copolymer of ethylene oxide and propylene oxide, lecithin, sodium dioctyl sulfosuccinate, sodium lauryl sulfate, Polysorbate 20, 60 & 80, Sorbitan esters, Sorbitan Fatty Acids Triton X-200, polyethylene glycol, glyceryl monostearate, d-alpha-tocopheryl polyethylene glycol 1000 succinate, sucrose fatty acid esters, sucrose stearate, sucrose oleate, sucrose palmitate, sucrose laurate, sucrose acetate butyrate, or mixtures thereof.

10. The composition according to claim 9 wherein the surfactant is present in an amount of 0 to about 15% w/w.

11. The composition according to claim 1 wherein the composition further comprises an absorption enhancer.

12. The composition according to claim 1 which provides a taste masking effect of the active agent.

13. The composition according to claim 1 wherein the polymeric carrier is polyvinyl alcohol, polyvinyl acetate, polyvinyl pyrrolidone, hyaluronic acid, alginates, carragenen, cellulose derivatives such as carboxymethyl cellulose sodium, methyl cellulose, ethylcellulose, hydroxyethyl cellulose, hydroxypropylcellulose, hydroxypropylmethyl cellulose, hydroxypropylmethyl cellulose phthalate, cellulose acetate phthalate, noncrystalline cellulose, starch and its derivatives such as hydroxyethyl starch, sodium starch glycolate, chitosan and its derivatives, albumen, gelatin, collagen, polyacrylates and its derivatives, poly(alpha-hydroxy acids), poly(alpha-aminoacids) and its copolymers, poly(orthoesters), polyphosphazenes, or poly(phosphoesters).

14. The composition according to claim 13 wherein the polymeric carrier is polyvinyl pyrrolidone or polyvinylpyrrolidone-co-polyvinylacetate.

15. The composition according to claim 13 wherein the polymeric carrier is Eudragit L100-55, Eudragit L30 D55, Eudragit L100, Eudragit S 100, Eudragit E 100, Eudragit EPO, Eudragit RL 30D, Eudragit RL PO, Eudragit RL 100, Eudragit RS 30D, Eudragit RS PO, Eudragit RS 100, Eudragit NE 30, or Eudragit NE 40, or a mixture thereof.

16. The composition according to claim 1 wherein said drug substance is an analgesic, anti-inflammatory agent, anthelmintic, anti-arrhythmic agent, an antibiotic, anticoagulant, antidepressant, antidiabetic agent, antiepileptic, antihistamine, antihypertensive agent, antimuscarinic agent, antimycobacterial agent, antineoplastic agent, immunosuppressant, antithyroid agent, antiviral agent, anxiolytic sedative, astringent, beta-adrenoceptor blocking agent, contrast media, corticosteroid, cough suppressant, diuretic, dopaminergic, homeostatic, immunological agent, lipid regulating agent, muscle relaxant, parasympathomimetic, parathyroid, calcitonin, prostaglandin, radio-pharmaceutical, sex hormone, steroid, anti-allergic agent, antihistaminic, stimulant, sympathomimetic, thyroid agent, vasodilator, PDE IV inhibitor, or a mixture thereof.

17. The composition according to claim 1 wherein the drug substance is aspirin, (S)-3-Hydroxy-2-phenyl-N-(1-phenylpropyl)-4-quinolinecarboxamide; 6-Acetyl-3,4-dihydro-2,2-dimethyl-trans(+)-4-(4-fluorobenzoylamino)-2H-benzo[b]pyran-3-ol hemihydrate, Rosiglitazone, Carvedilol, Eposartan, hydrochlorthiazide, nifedipine, ketoprofen, indomethacin, (3R,3aS,6aR)-hexahydrofuro[2,3-b]furan-3-yl (1S,2R)-3-[(1,3-benzodioxol-5-ylsulfonyl)(isobutyl)amino]-2-hydroxy-1-{4-[(2-methyl-1,3-thiazol-4-yl)methoxy]benzyl}propylcarbamate, or a pharmaceutically acceptable salt thereof of any of these agents.

18. The composition according to claim 1 in which active agent is present in an amount of about 1 to about 50% w/w.

19. The composition according to claim 1 which is intended for oral administration.

20. The composition according to claim 1 in which the active agent demonstrates improved bioavailability and/or improved stability, or has a modified or delayed absorption profile as compared to an immediate release dosage form.

21. The composition according to claim 1 in which the electrospun fiber is encapsulated or compressed into a tablet or capsule.

22. The composition according to claim 1 in which the electrospun fiber is further ground in size.

23. The composition according to claim 1 which is results in a rapid dissolution of the fiber.

24. The composition according to claim 1 which results in controlled release, sustained release, or pulsatile release of the active agent.

25. The composition according to claim 1 which results in immediate release of the active agent.

26. Use of a composition according to claim 1 for inhalation therapy.

27. Use of a composition according to claim 1 for dispersion in an aqueous solution.

28. A process for making a stable formulation of an amphorous form of a pharmaceutically active agent comprising
a) making a solution of the active agent, and a pharmaceutically acceptable polymeric carrier with a pharmaceutically acceptable solvent; and
b) electrospinning the solution of step (a) into an electrospun fiber.

29. The process according to claim 28 wherein the solvent is water miscible.

30. The process according to claim 28 wherein the solvent is water immisicible.

31. The process according to claim 28 wherein the solution is mixture of one or more solvents.

32. The process according to claim 29 wherein the solvent is a mixture of water and a water miscible solvent.

33. The process according to claim 28 wherein the solvent is ethanol, or a mixture of ethanol and methylene chloride or tetrahydrofuran.

34. The process according to claim 28 wherein the polymeric carrier is polyvinyl alcohol, polyvinyl acetate, polyvinyl pyrrolidone, hyaluronic acid, alginates, carragenen, cellulose derivatives such as carboxymethyl cellulose sodium, methyl cellulose, ethylcellulose, hydroxyethyl cellulose, hydroxypropylcellulose, hydroxypropylmethyl cellulose, hydroxypropylmethyl cellulose phthalate, cellulose acetate phthalate, noncrystalline cellulose, starch and its derivatives such as hydroxyethyl starch, sodium starch glycolate, chitosan and its derivatives, albumen, gelatin, collagen, polyacrylates and its derivatives, poly(alpha-hydroxy acids) and its copolymers such poly(caprolactone), poly(alpha-aminoacids) and its copolymers, poly(orthoesters), polyphosphazenes, or poly(phosphoesters).

35. The process according to claim 34 wherein the polymeric carrier is polyvinyl pyrrolidone, or polyvinylpyrrolidone-co-polyvinylacetate.

36. The composition according to claim 34 wherein the polymeric carrier is Eudragit L100-55, Eudragit L30 D55, Eudragit L100, Eudragit S 100, Eudragit E 100, Eudragit EPO, Eudragit RL 30D, Eudragit RL PO, Eudragit RL 100, Eudragit RS 30D, Eudragit RS PO, Eudragit RS 100, Eudragit NE 30, or Eudragit NE 40, or a mixture thereof.

37. The process according to claim 28 wherein the active agent is an analgesic, anti-inflammatory agent, anthelmintic, anti-arrhythmic agent, an antibiotic, anticoagulant, antidepressant, antidiabetic agent, antiepileptic, antihistamine, antihypertensive agent, antimuscarinic agent, antimycobacterial agent, antineoplastic agent, immunosuppressant, antithyroid agent, antiviral agent, anxiolytic sedative, astringent, beta-adrenoceptor blocking agent, contrast media, corticosteroid, cough suppressant, diuretic, dopaminergic, homeostatic, immunological agent, lipid regulating agent, muscle relaxant, parasympathomimetic, parathyroid, calcitonin, prostaglandin, radio-pharmaceutical, sex hormone, steroid, anti-allergic agent, antihistaminic, stimulant, sympathomimetic, thyroid agent, vasodilator, PDE IV inhibitor, or a mixture thereof.

38. The composition according to claim 28 wherein the active agent is aspirin, (S)-3-Hydroxy-2-phenyl-N-(1-phenylpropyl)-4-quinolinecarboxamide, or 6-Acetyl-3,4-dihydro-2,2-dimethyl-trans(+)-4-(4-fluorobenzoylamino)-2H-benzo[b]pyran-3-ol hemihydrate, Rosiglitazone, Carvedilol, Eposartan, hydrochlorthiazide, nifedipine, ketoprofen, or indomethacin.

39. The product produced by the process according to claim 28.

40. A process for making a stable formulation of an amphorous form of a pharmaceutically active agent comprising
a) melting the active agent and a pharmaceutically acceptable polymeric carrier to form a melt; and
b) electrospinning the melt of step (a) into an electrospun fiber.

41. The process according to claim 40 wherein the polymeric carrier is polyvinyl alcohol, polyvinyl acetate, polyvinyl pyrrolidone, hyaluronic acid, alginates, carragenen, cellulose derivatives such as carboxymethyl cellulose sodium, methyl cellulose, ethylcellulose, hydroxyethyl cellulose, hydroxypropylcellulose, hydroxypropylmethyl cellulose, hydroxypropylmethyl cellulose phthalate, cellulose acetate phthalate, noncrystalline cellulose, starch and its derivatives such as hydroxyethyl starch, sodium starch glycolate, chitosan and its derivatives, albumen, gelatin, collagen, polyacrylates and its derivatives, poly(alpha-aminoacids) and its copolymers, poly(orthoesters), polyphosphazenes, or poly(phosphoesters).

42. The process according to claim 41 wherein the polymeric carrier is polyvinyl pyrrolidone, or polyvinylpyrrolidone-co-polyvinylacetate.

43. The composition according to claim 41 wherein the polymeric carrier is wherein the polymeric carrier is Eudragit L100-55, Eudragit L30 D55, Eudragit L100, Eudragit S 100, Eudragit E 100, Eudragit EPO, Eudragit RL 30D, Eudragit RL PO, Eudragit RL 100, Eudragit RS 30D, Eudragit RS PO, Eudragit RS 100, Eudragit NE 30, or Eudragit NE 40, or a mixture thereof.

44. The process according to claim 41 wherein the active agent is an analgesic, anti-inflammatory agent, anthelmintic, anti-arrhythmic agent, an antibiotic, anticoagulant, antidepressant, antidiabetic agent, antiepileptic, antihistamine, antihypertensive agent, antimuscarinic agent, antimycobacterial agent, antineoplastic agent, immunosuppressant, antithyroid agent, antiviral agent, anxiolytic sedative, astringent, beta-adrenoceptor blocking agent, contrast media, corticosteroid, cough suppressant, diuretic, dopaminergic, homeostatic, immunological agent, lipid regulating agent, muscle relaxant, parasympathomimetic, parathyroid, calcitonin, prostaglandin, radio-pharmaceutical, sex hormone, steroid, anti-allergic agent, antihistaminic, stimulant, sympathomimetic, thyroid agent, vasodilator, PDE IV inhibitor, or a mixture thereof.

45. The composition according to claim 41 wherein the active agent is, aspirin, (S)-3-Hydroxy-2-phenyl-N-(1-phenylpropyl)-4-quinolinecarboxamide, or 6-Acetyl-3,4-dihydro-2,2-dimethyl-trans(+)-4-(4-fluorobenzoylamino)-2H-benzo[b]pyran-3-ol hemihydrate, Rosiglitazone, Carvedilol, Eposartan, hydrochlorthiazide, nifedipine, ketoprofen or indomethacin.

46. The product produced by the process according to claim 41.
